(19) United States
(12) Patent Application Publication
(10) Pub. No.: US 2013/0104881 A1
(43) Pub. Date: May 2, 2013

(54) Stabilized Metered Dose Inhaler

(71) Applicants: Fernando Toneguzzo, Buenos Aires, AR (US); Julio Cesar Vega, Buenos Aires (AR)

(72) Inventors: Fernando Toneguzzo, Buenos Aires, AR (US); Julio Cesar Vega, Buenos Aires (AR)

(73) Assignee: Laboratorio Pablo Cassara S.R.L., Buenos Aires (AR)

(21) Appl. No.: 13/286,171

(22) Filed: Oct. 31, 2011

(51) Int. Cl. A61M 11/00; A61K 31/137; A61K 31/573; A61P 11/00; A61K 31/196; A61K 31/439; A61P 11/08; A61K 9/72; A61K 31/58

(52) U.S. Cl. 128/200.23; 424/400; 514/653; 514/180; 514/174; 514/564; 514/299; 514/291

(57) ABSTRACT

Pharmaceutical pressurized metered dose inhalers are disclosed having improved temperature and moisture stability comprising CFC-free, ethanol-free compositions that include lactose of particle diameter larger than 1 μm as a means of minimizing sticking to the walls and other destabilization mechanisms activated by moisture ingress from the environment.

The present invention relates to the discovery of an innovative way of stabilizing pressurized metered dose inhalers against environments ranging from standard storage (23 to 32° C./55-75% RH) and during stress conditions (40° C./RH 75% RH). The disclosed compositions have non-CFC propellants. thus allowing the formulation of commercial products without need of protecting them towards high humidity.

Inhalation has become a widely used route of administration of bronchodilators, steroids and other medications to the airways of patients suffering from respiratory diseases. One of the pharmaceutical dosage forms used for this purpose are pressurized metered dose inhalers (MDIs).

Pressurized metered dose inhalers need a propellant in their formulation to produce a fine spray of micronized particles as the formulation is expelled through the valve stem and the actuator orifice into the oral cavity of the patient. Until recently, the most widely used propellants for this purpose were chlorofluorocarbons (CFCs). However, it has been discovered that these propellants react with the ozone layer of the atmosphere and contribute to its depletion. Recently, FDA and most of the governments of the world have begun to ban their use even for pressurized metered-dose inhalers. Therefore, in the past years several efforts have been made by pharmaceutical companies to re-formulate their products into non-ozone depleting formulations, mainly by replacing CFCs by hydrofluoroalkanes (HFAs). The most widely used HFAs for this purpose have been HFA 134a (Norflurane or 1,1,1,2-tetrafluoroethane) and HFA 227ea as can be illustrated by the teachings of Purewal et al. (U.S. Pat. No. 5,776,434) and Akehurst et al., (U.S. Pat. No. 6,893,628). Hydrocarbon formulations have also been disclosed by Warnke et al., (WO 93/06185).

Active ingredients have been formulated in suspension (e.g., Purewal et al. U.S. Pat. No. 5,776,434), solution (e.g. U.S. Pat. No. 6,045,778) and a combination of active ingredients in suspension and others in solution (e.g. U.S. Pat. No. 6,423,298). Several formulations with different active ingredients with the use of different excipients have been extensively studied and taught through patents. However, several HFA MDIs must be packaged into pouches with desiccants to protect them from humidity as is the case with the following marketed products in the United States:

Advair (fluticasone propionate+Salmeterol Xinafoate)

Flovent (fluticasone propionate)

Symbicort (Budesonide+Formoterol Fumarate)

Ventolin (Salbutamol Sulphate)

Some formulations that include ethanol in their formulation do not need any protection against humidity. This is the case of the marketed products Proventil HFA and Proair. However, inclusion of ethanol may be disadvantageous because:

Alcoholic taste and smell is not tolerated well by some

Some people could not use them because of religious reasons (e.g., Muslims)

Some people could prefer not to use them (e.g., alcoholics, children)

Partial dissolution of suspended active ingredient which could give rise to crystal growth, increased bitterness and chemical degradation of the dissolved active ingredient during shelf life

Physically stable ethanol-free suspensions (MDIs) resistant to high humidity are not found in the prior art and present an unmet need that the present invention fills. It is noteworthy that a large number of countries around the world have high humidity including the US, Brazil, Mexico, Colombia, South Africa, India, China, Italy, Spain, France, etc.

### BRIEF DESCRIPTION OF THE INVENTION

Surprisingly, the inclusion of lactose with a mean particle size larger than 1 μm allows the formulation of suspension MDIs with markedly increased resistance against humidity, without the inclusion of ethanol in the formulation.

### DETAILED DESCRIPTION OF THE INVENTION

It has been surprisingly discovered that the addition of small amounts of lactose having a mass median particle size larger than 1 μm allows the formulation of suspension MDIs particularly resistant to humid environments such as those encountered in the so called climatic zone II, IVa and IVb countries. For regulatory purposes the world is divided in four or five climatic zones for the performance of stability studies of medicines:

Climatic Zone I (UK, Germany, Sweden, etc.): 21° C./45% RH

Climatic Zone II (US, Mexico, Spain, Italy, France, Argentina, etc.): 25° C./60% RH

Climatic Zone III (Saudi Arabia, Egypt, etc.): 30° C./45% RH

Climatic Zone IV a (Colombia, Peru, etc.): 30° C./65% RH (tropical countries)

Climatic Zone IV b (Brazil): 30° C./ 75% RH (tropical countries)

While not wishing to be limited to any particular theory, it is believed that addition of lactose works as an alternative surface for the adsorption of some active ingredients otherwise showing adhesion to the walls in humid environments, most probably due to moisture ingress. Thus, these active ingredients seem to adsorb themselves reversibly onto lactose particles. Adsorption forces are weak enough to allow the active ingredient to leave the surface of lactose when being exerted by the flash vaporization of propellant through the orifice of the actuator, when the device is actuated. That means that even if adsorption took place, it does not prevent the active ingredients from reaching high percentage of fine particles, as demonstrated below in Example 9.

It is important to point out that this anti-adhering effect of lactose has not been disclosed before in the formulation of pressurized metered dose inhalers. Only the potential of submicronic lactose as bulking agent to improve re-dispersibility of suspensions has been taught in US Patent Application 20090246149. In the present invention, the inclusion of lactose having a much larger particle size than that disclosed as bulking agent in prior art, has surprisingly resulted in less adhesion to the walls of active ingredients, such as formoterol fumarate dihydrate and salmeterol xinafoate. In addition, there are several other active ingredients that tend to stick to the can wall such as fluticasone propionate in low doses (50 μg) and other corticosteroids. Lactose having a particle size with a mass median diameter larger than 1 μm, i.e., preferably having more than 50% of the mass above 1 μm, can be added to avoid sticking to the wall and most surprisingly without producing clogging of the valve or actuator orifice, even with very small orifice diameters (0.25 mm). Most surprisingly yet, the formulation need not include ethanol as co-solvent at all. This is a further advantage because the addition of an aliphatic alcohol, though useful to dissolve excipients and active ingredients and so far practically unavoidable in formulating humidity resistant pressurized metered dose inhalers has many disadvantages ad presented above.

In all embodiments, lactose having a mass median diameter larger than 1 μm is included in lactose in a total mass ratio of 100/1 to 1/1. Lactose used for this purpose should have a low surface free enthalpy, i.e., it is preferably obtained by milling or sieving without the use of high-energy air jet micronization.

In all embodiments, the propellants used are hydrocarbons, volatile ethers and/or hydrofluoroalkanes. Hydrofluoroalkanes can be selected from the group: Norflurane (1,1,1,2-Tetrafluoroethane, also called HFA 134a), HFA 227ea (1,1,1,2,3,3,3-heptafluoropropane) or others known in the art. Hydrocarbons can be selected from the group: isobutane, propane, n-butane, n-pentane or others known in the art. Volatile ether used is dimethylether or others known in the art.

In some embodiments suspension stabilizers such as oleic acid, sorbitan trioleate, lecithin, polyethylene glycol, povidone or other known in the art can be used.

In all embodiments, a suitable amount of a pharmaceutically active ingredient is added to render the correct dose when a puff is released from the valve-metering chamber. Active ingredients could be: salbutamol, salbutamol sulphate, beclomethasone dipropionate, budesonide, formoterol fumarate, fluticasone propionate, fluticasone fumarate, mometasone furoate, salmeterol xinafoate, ciclesonide, ipratropium bromide, oxitropium bromide, tiotropium bromide and their salts as well as other therapeutically active substances suitable to be administered by inhalation.

In those embodiments where the active ingredient is suspended, it should be micronized so that 100% of the particles are below 20 μm in diameter and 95% of the particles are below 10 μm.

In all embodiments the formulation is packaged into cans fitted with a metering valve.

### EXAMPLE 1

This example illustrates the adequate flocculation/sedimentation characteristics obtained using lactose of the characteristics described in the detailed description of the invention without the need of using Ethanol. The amount of fluticasone propionate corresponds to the therapeutic dose using a 50 microliter metering valve.

| Ingredient | % w/w | % w/w |
|---|---|---|
| Fluticasone propionate | 0.41 | 0.20 |
| Lactose | 0.16 | 0.15 |
| Polyethylene glycol | 0.20 | 0.16 |
| Norflurane | q.s. 100 | q.s. 100 |

### EXAMPLE 2

The following composition forms a stable ethanol-free suspension of fluticasone propionate and salmeterol xinafoate with suitable flocculation and sedimentation characteristics for inhalation, using lactose in the composition.

| Ingredient | % w/w |
|---|---|
| Fluticasone propionate | 0.20 |
| Salmeterol Xinafoate | 0.06 |
| (Hydroxynaphthoate) | |
| Lactose | 0.16 |
| Polyethylene glycol | 0.17 |
| Norflurane | q.s. 100 |

### EXAMPLE 3

The following composition forms a stable ethanol-free suspension of Salbutamol Sulfate with suitable flocculation and sedimentation characteristics for inhalation, using lactose in the composition.

| Ingredient | % w/w |
|---|---|
| Salbutamol Sulfate | 0.2 |
| lactose | 0.16 |
| Polyethylene glycol | 0.13 |
| Norflurane | q.s. 100 |

### EXAMPLE 4

The following composition forms a stable ethanol-free suspension of Salbutamol Sulfate and Ipratropium Bromide with suitable flocculation and sedimentation characteristics for inhalation, using lactose in the composition.

| Ingredient | % w/w |
|---|---|
| Salbutamol Sulfate | 0.2 |
| Ipratropium Bromide | 0.034 |
| mono-hydrate | |
| Lactose | 0.16 |
| Polyethylene glycol | 0.15 |
| Norflurane | q.s. 100 |

### EXAMPLE 5

The following composition forms a stable ethanol-free suspension of Ipratropium Bromide with suitable flocculation and sedimentation characteristics for inhalation, using lactose in the composition.

| Ingredient | % w/w |
|---|---|
| Ipratropium Bromide mono- | 0.034 |
| hydrate | |
| lactose | 0.07 |
| Polyethylene glycol | 0.12 |
| Norflurane | q.s. 100 |

### EXAMPLE 6

The following composition forms a stable ethanol-free suspension of Beclomethasone Dipropionate with suitable flocculation and sedimentation characteristics for inhalation, using lactose in the composition.

| Ingredient | % w/w |
|---|---|
| Beclomethasone Dipropionate | 0.08 |
| lactose | 0.10 |
| Polyethylene glycol | 0.1 |
| Norflurane | q.s. 100 |

### EXAMPLE 7

The following composition forms a stable ethanol-free suspension of salbutamol sulfate and beclomethasone dipropionate with suitable flocculation and sedimentation characteristics for inhalation, using lactose in the composition.

| Ingredient | Percentage amount |
|---|---|
| Salbutamol Sulfate | 0.20 |
| Beclomethasone | 0.08 |
| dipropionate | |
| lactose Respitose ML 006 | 0.13 |
| Polyethylene glycol | 0.15 |
| Norflurane | q.s. 100 |

### EXAMPLE 8

The following composition forms a stable ethanol-free suspension of Budesonide with suitable flocculation and sedimentation characteristics for inhalation, using lactose in the composition.

| Ingredient | % w/w |
|---|---|
| Budesonide | 0.32 |
| Lactose | 0.18 |
| Polyethylene glycol | 0.12 |
| Norflurane | q.s. 100 |

### EXAMPLE 9

This example shows that ethanol-free suspensions obtained using lactose with mass median diameter larger than 1 μm are resistant against humidity and this is due to the addition of lactose to the formulation.

TABLE 1
Composition of the different formulations tested in % w/w

| Ingredient | A | B | C |
|---|---|---|---|
| Budesonide | 0.295 | 0.295 | 0.295 |
| Formoterol | 0.0083 | 0.0083 | 0.0083 |
| Polyethylene glycol | 0.1 | 0.1 | 0.3 |
| Povidone | 0 | 0 | 0.00027 |
| HFA 134a | 99.4 | 99.6 | 99.4 |
| lactose | 0.163 | 0 | 0 |

TABLE 2
Performance of formulations A, B and C tested at an initial period of
time (I) and after 6 months at 40° C./75% RH and 6 months
at 30° C./75% RH.

| | | Budesonide | Formoterol | Water |
|---|---|---|---|---|
| Formulation | | Assay | Assay | (μg/g) |
| A | I | 100 | 100 | 524 |
| | 6 months at | 112.9* | 103.6* | 546.2 |
| | 40° C. 75% | | | |
| | RH | | | |
| | 6 months at | 109.3* | 103.6* | 530.4 |
| | 30° C. 75% | | | |
| | RH | | | |
| B | I | 100 | 100 | 100.2 |
| | 6 months at | 75.1* | 75.7* | 1403.4 |
| | 40° C. 75% | | | |
| | RH | | | |
| | 6 months at | 92.4* | 96.9* | 1601.7 |
| | 30° C. 75% | | | |
| | RH | | | |
| C | I | 100 | 100 | 116.1 |
| | 6 months at | 106.4* | 75.4* | 1331.5 |
| | 40° C. 75% | | | |
| | RH | | | |
| | 6 months at | 113.3* | 87.5* | 1115.2 |
| | 30° C. 75% | | | |
| | RH | | | |

All values except water are expressed as percentages.
*Results are expressed as percentage of initial (I) value under the respective condition.

The above results indicate that the water content is fixed at a certain amount in the lactose containing formulations, probably due to hydration water of lactose monohydrate, whereas the other formulations present growing water contents throughout storage at highly humid environments. The water content increase in the non-lactose containing formulation is most likely composed of free water, which could interact with the suspended active ingredients and increase their tendency to adhere to the container walls.

Additionally, no clogging was observed in any unit after being actuated 300 times. This effect could be explained on the basis of a relatively low surface free enthalpy due to the use of sieving or low energy milling instead of air jet micronization to reduce the particle size of lactose.

The following results (Table 3) of deposition of the emitted dose obtained in formulation A demonstrate that lactose-containing formulation allows adequate deposition of the suspended active ingredients (expected value for both active ingredients: not less than 25% particles below 6.4 μm as determined in Apparatus A of European Pharmacopeia using an air flow of 60 Liter/minute).

TABLE 3
Deposition of the emitted dose

| | | Budesonide | Formoterol |
|---|---|---|---|
| Formulation | | Deposition | Deposition |
| A | I | 33.10% | 42.70% |
| | 6 months at | 32.30% | 41.60% |
| | 40° C. 75% RH | | |
| | 6 months at | 34.20% | 44.70% |
| | 30° C. 75% RH | | |

Additionally, fine particle fraction was determined on formulation A using Andersen Cascade Impactor. A relatively large fine particle fraction confirms that lactose does not prevent the active ingredients from reaching high percentage of fine particles when the aerosol is actuated.

TABLE 4
Fine particle fraction recovery

Recoveries (%)

| Step | μm | Formoterol | Budesonide |
|---|---|---|---|
| PI | — | 32.5 | 41.8 |
| 0 | 9.00-10.00 | 1.1 | 1.4 |
| 1 | 5.80-9.00 | 2.7 | 3.2 |
| 2 | 4.70-5.80 | 3.9 | 5.7 |
| 3 | 3.30-4.70 | 7.5 | 10.5 |
| 4 | 2.10-3.30 | 20.8 | 20.2 |
| 5 | 1.10-2.10 | 21.6 | 13.7 |
| 6 | 0.65-1.10 | 10.6 | 4.4 |
| 7 | 0.43-0.65 | 0.9 | 0.5 |
| F | — | 1.2 | 1.3 |

| | | |
|---|---|---|
| Total | 102.9 | 102.7 |
| FPF (EuPh) | 63.7 | 52.2 |

What is claimed is:

1. A pharmaceutical pressurized metered dose inhaler containing a liquid suspension comprising:
a. At least one pharmaceutically active ingredient;
b. At least one non-chlorofluorcarbon (non-CFC) propellant;
c. Lactose powder of a median diameter larger than 1 μm and in a concentration ranging between 0.01 and 2% w/w of the liquid suspension,
d. At least one suspension stabilizer selected from a group consisting of polyethylene glycols, oleic acid, sorbitan trioleate, povidone, poloxamers and a combinations thereof.

2. The composition according to claim 1, wherein the stabilizer is polyethylene glycol in a concentration ranging between 0.01 and 0.5% w/w of the liquid suspension.

3. The composition according to claim 1, wherein said composition is packaged in cans fitted with a metering valve delivering between 20 and 200 microliter of formulation per shot.

4. The composition according to claim 1, wherein said propellant comprises a hydrocarbon, hydrofluorocarbon or ether.

5. The composition according to claim 1, wherein said active ingredient is selected from a group of drugs administered by inhalation consisting of salbutamol, salbutamol sulphate, beclomethasone dipropionate, budesonide, formoterol fumarate, fluticasone propionate, fluticasone fumarate, mometasone furoate, salmeterol xinafoate, ciclesonide, ipratropium bromide, oxitropium bromide, tiotropium bromide and their salts.

6. The composition according to claim 1, wherein the active ingredient is budesonide.

7. The composition according to claim 1, wherein the active ingredient is formoterol fumarate dihydrate or a salt or hydrate thereof

8. The composition according to claim 1, wherein the active ingredient are budesonide and formoterol fumarate dihydrate or a salt or hydrate thereof.

9. The composition according to claim 2, wherein said active ingredient is selected from a group of drugs administered by inhalation route consisting of salbutamol, salbutamol sulphate, beclomethasone dipropionate, budesonide, formoterol fumarate, fluticasone propionate, fluticasone fumarate, mometasone furoate, salmeterol xinafoate, ciclesonide, ipratropium bromide, oxitropium bromide, tiotropium bromide and their salts thereof.

10. The composition according to claim 2, wherein the active ingredient is budesonide.

11. The composition according to claim 2, wherein the active ingredient is formoterol fumarate dihydrate or a salt or hydrate thereof.

12. The composition according to claim 2, wherein the active ingredient is a budesonide and formoterol fumarate dihydrate or a salt or hydrate thereof.
